(19) United States
(12) Patent Application Publication
(10) Pub. No.: US 2008/0281250 A1
(43) Pub. Date: Nov. 13, 2008

(54) Self-Clearing Catheter for Clinical Implantation

(76) Inventors: Marvin Bergsneider, Los Angeles, CA (US); Selene Lee, Los Angeles, CA (US); Jack W. Judy, Los Angeles, CA (US)

Correspondence Address: GATES & COOPER LLP;HOWARD HUGHES CENTER, 6701 CENTER DRIVE WEST, SUITE 1050, LOS ANGELES, CA, 90045, US

(21) Appl. No.: 11/919,936

(22) PCT Filed: May 10, 2006
(86) PCT No.: PCT/US2006/018026

§ 371 Date: Nov. 6, 2007

Related U.S. Application Data
(60) Provisional application No. 60/679,350, filed on May 10, 2005.

(51) Int. Cl. A61M 5/00

(52) U.S. Cl. 604/9

(57) ABSTRACT

The invention disclosed herein provides methods and apparati that utilize micro-electro-mechanical systems that can be used for example to prevent and/or clear obstructions in the fluid conduits of medical devices such as catheters.

### RELATED APPLICATIONS

This application claims the benefit under 35 U.S.C. §119(e) of Provisional Patent Application Ser. No. 60/679,350 filed on May 10, 2005, the contents of which are incorporated by reference.

### STATEMENT OF GOVERNMENT SUPPORT

This invention was made with Government support from NSF-DGE-997280 from the National Science Foundation. The Government may have certain rights to this invention.

### FIELD OF THE INVENTION

The present invention relates to methods and apparati used with medical devices having fluid conduits such as catheters.

### BACKGROUND OF THE INVENTION

A wide variety of implantable medical devices include fluid conduits such as catheters that require adequate fluid flow (e.g. the fluid conduit remaining open) for optimal functioning. Catheter occlusions that inhibit fluid flow can lead to device failure and can produce life-threatening situations for a patient. Eventually, a malfunctioning device typically requires implant replacement, a factor that increases a patient's risk of infection and surgical complications. While a number of catheter designs which endeavor to avoid and/or prevent occlusions have been tested over several decades, catheter occlusion remains a significant problem in clinical practice.

One exemplary condition treated with implantable medical devices having fluid conduits is hydrocephalus. It is estimated that one in every 500 newborns is afflicted by this condition, which is characterized by an abnormal accumulation of cerebrospinal fluid (CSF) within the fluid cavities of the brain. The mainstay of treatment of hydrocephalus is an implantable device, called a shunt, which diverts CSF to another part of the body. A CSF shunt typically has three components: a CSF access catheter (generally a “ventricular” catheter), a valve mechanism to regulate fluid flow, and a distal catheter placed in a part of the body where the CSF is deposited.

Over 25,000 CSF shunt operations are completed each year in the U.S. alone. Patients who receive implanted shunts are dependent on the device functioning properly. Unfortunately, this device, which is so critical to managing hydrocephalus, has a substantial failure rate. A malfunctioning (e.g. obstructed) shunt can be a life-threatening condition. The most common part of the shunt that becomes obstructed is the proximal (ventricular) catheter. On average, 85% of people with shunts have at least two shunt-revision surgeries in their lifetime. A minority of patients are plagued with recurrent shunt obstructions and may undergo over 100 shunt revisions. Shunt-replacement surgeries can cause temporary and sometimes significant morbidity. Each successive shunt revision may cause brain injury and increase the risk of shunt infection.

A number of approaches designed to prevent and/or clear obstructions in fluid conduits of medical devices such as CSF shunts have been considered in the art. For example, for hydrocephalus, a variety of ventricular catheter designs have been used (e.g. slots, flanges, small versus larger holes, etc.). In addition, approaches using various designs and/or materials adapted to address these issues are also known in the art. The use of a percutaneously inserted laser fiberoptic lead for clearing an obstructed catheter has also been used clinically. Catheters can also, for example, use one or more hydrophilic surface layers to absorb antibiotic solutions and repel protein adhesion to the catheter. With intravascular catheters, artisans have also considered heparin-coated catheters and/or intermittent instillation of heparinized fluid.

### SUMMARY OF THE INVENTION

The invention disclosed herein is designed for use with medical devices in which it is desirable to facilitate and/or modulate the flow of a fluid through a fluid conduit of the medical device. The invention disclosed herein has a variety of embodiments. An illustrative embodiment is an implantable medical device comprising a fluid conduit having an orifice; and an actuator coupled to the orifice of the fluid conduit; wherein the actuator alternates between a first and second position in response to a signal so as to inhibit the accumulation of materials and/or prevent the obstruction at the orifice of the fluid conduit.

In some embodiments of the invention, the fluid conduit is adapted for use as a shunt for use in the treatment of hydrocephalus. In a specific illustrative embodiment of the invention, magnetic actuators are used to allow their easy integration into a catheter that can easily integrate with existing implantable medical devices. In one such embodiment, the actuator devices are activated by an external magnetic field that guides the magnetic actuators to maintain specific physical positions. In some embodiments, the design of the device is such that it is safe for use in strong magnetic fields such as magnetic resonance imaging (MRI).

An illustrative embodiment of the invention is an implantable medical device comprising a fluid conduit having an orifice, and an actuator coupled to the orifice of the fluid conduit, wherein the actuator alternates between a first and a second position in response to a signal so as to inhibit accumulation of materials or remove materials at the orifice of the fluid conduit. Typically, the actuatable member at the orifice of the fluid conduit moves in such a manner to prevent or reduce occlusion of the orifice of the fluid conduit. In certain embodiments of the invention, the actuator restricts fluid flow through the fluid conduit. Optionally, the fluid conduit is adapted for use as a shunt such as a shunt is adapted for use in the treatment of hydrocephalus. In certain embodiments of the invention, the medical device is coated with a composition that further inhibits the accumulation of materials at the orifice of the fluid conduit. Optionally, a composition of the actuator is selected to be compatible with strong magnetic fields such as MRI. In certain embodiments of the invention, the signal is a remote signal, for example a remote magnetic signal comprises an external oscillating magnetic field.

Another embodiment of the invention is a method of removing materials or decreasing materials that accumulate at an orifice of a fluid conduit of an implantable medical device by using an external signal to actuate an actuatable member disposed at the orifice of the fluid conduit, wherein the actuatable member alternates between a first and a second position in response to the external signal so that the materials are removed or the accumulation of materials at the orifice of the fluid conduit is decreased. Typically, the actuator alternates in a manner comprising a sweeping, vibrating, or rotating motion. In illustrative methods of the invention, the signal comprises an external magnetic signal. In certain embodiments of the invention, a device that generates the signal is placed in proximity to the head of a patient.

Yet another embodiment of the invention is a method of manufacturing a fluid conduit having an actuator for use in an implantable medical device comprising providing a base substrate of the fluid conduit, coupling an actuator to the substrate of the fluid conduit. In this method, the actuator is coupled to the fluid conduit so as to allow the actuator to alternate between a first and a second position in response to a signal in a manner that inhibits accumulation of materials or remove materials at an orifice of the fluid conduit when the medical device is implanted in vivo. In certain methods, the actuator is manufactured using microelectromechanical systems (MEMS) techniques.

Embodiments of the invention also include articles of manufacture and/or kits designed for example to facilitate the methods of the invention. Typically such kits include instructions for using the implantable medical devices and/or actuator elements within the kit according to the methods of the present invention. Such kits can comprise a carrier means being compartmentalized to receive in close confinement one or more container means such as vials, tubes, and the like, each of the container means comprising one of the separate elements to be used in methods of the invention.

### BRIEF DESCRIPTION OF THE DRAWINGS

FIG. 1 provides a schematic view of a typical embodiment of an actuator 20 within an orifice of a fluid conduit 10. In this embodiment, the actuator(s) 20 comprise a moveable actuatable member 30 connected to a surrounding frame 40 by bridging element 50. As shown in this figure, an implantable medical device may comprise one or more actuators 20. The actuators 20 may be separately integrated within the catheter material or manufactured in a neighboring configuration so that multiple actuators can coexist on a single surface, termed an actuator complex. The actuators 20 or actuator complexes can be situated at or near the terminus of the catheter, or anywhere along the path of the fluid conduit as single or multiple units and/or complexes.

FIG. 2 provides an enlarged view of a typical embodiment of an actuator within an orifice of a fluid conduit. The bridging element 50 is the mechanical link between the moveable element 30 and the surrounding frame 40. The characteristics of the bridging element are 1) to provide an attachment point(s) of the moveable element 30 to the surrounding frame 40, 2) to return the moveable element 30 to its resting baseline position if not being deflected by the external magnet forces, 3) to allow sufficient movement of the moveable element 30 in order to fulfill the requirements of freeing or deterring biological/chemical occluding material, and 4) to prevent excessive movement of the moveable element 30 in its normal use or if submitted to large magnetic fields (such as an MRI).

FIG. 3 provides an enlarged view of an alternative embodiment of an actuator within an orifice. In the present embodiment, the bridging element is a single cantilever beam 70 that is the mechanical link between the movable element 30 and the surrounding frame 40. The actuatable member is round rather than rectangular.

FIG. 4 provides a sectional view of the different positions of the actuator plate. In FIG. 4, the actuator incorporates integral magnetic components that cause the moveable element to alternate between a first position 110 and a second position 120 in response to an external moving magnet field. The movement of the actuatable member will inhibit the formation of occluding materials at the orifice of the fluid conduit. 130-150 illustrate another sectional view of an embodiment of the invention further showing the movement of the actuators in response to magnetic field vectors.

### DETAILED DESCRIPTION OF THE INVENTION

The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted. Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The terms “implantable medical device” are used herein according to their art accepted meaning and include the variety of implantable medical devices known in the art such as shunts, intravenous catheters and/or arterial catheters, drug administration devices such as infusion pumps and the like. Such implantable medical devices are typically used in a number of contexts such as the shunting of in vivo fluids, drug delivery; dialysis; urinary disorders and the like (see, e.g., U.S. Pat. Nos. 6,470,211, 6,997,899, 7,008,395 7,018,373, 7,011,646, 7,018,362, 7,020,516 and 7,022,109, the contents of which are incorporated by reference). Implantable medical devices can be used to direct and/or maintain fluid flow, pressure, and/or temperature. Shunts and associated catheters can be used, for example, for the treatment of cardiac problems, ascites (abnormal accumulation of fluid in the abdomen), ocular problems (e.g., glaucoma and lacrimal fluid build-up), hydrocephalus, urinary disorders, synovial fluid, and liver hypertension. Implanted medical devices can also be used for delivery of medications into brain parenchyma via convection-enhanced delivery. As another example, when properly functioning, shunt systems provide an effective manner of regulating CSF in hydrocephalus patients. Implantable medical devices can also be used for providing the acquisition of important physiological data from the body by placing, for example, sensors in blood vessels or chambers of the heart for cardiac monitoring (see, e.g., U.S. Pat. No. 7,031,772 and U.S. Pat. No. 7,031,771, the contents of which are incorporated by reference). Implantable medical devices can be entirely implanted within the body, or some part of the medical device implanted either temporarily or permanently.

The term “fluid conduit” is used according to its broadest meaning and simply refers to a channel, tunnel, lumen or the like through which a fluid, is conveyed. As is known in the art, fluid conduits can comprise additional elements such as valves and the like. Illustrative examples of fluid conduits include, but are not limited to medical tubing, shunts, catheters and the like. Catheters typically comprise a hollow, usually flexible tube that is used to drain and/or infuse a body cavity. Catheters can be single or multi-lumen catheters and be adapted for short-term or long-term use. As is known in the art, fluid conduits in implantable medical devices are not limited to a particular dimension and come in a variety of shapes and sizes depending upon their desired function. For example, fluid conduits such as shunts and catheters vary in diameter and thickness and can have one or more orifices of varying sizes (see, e.g., U.S. Patent Application 20050256510 and U.S. Patent Application 20040199109, the contents of which are incorporated by reference). Those of skill in the art understand that the actuators of the invention can consequently come in a variety of shapes and sizes depending upon the implantable medical device for which they are constructed.

The term “shunt” is used according to its broadest meaning and within the embodiment detailed, refers to a single conduit or a combination of conduits plus a valve mechanism, that diverts CSF from the brain or spinal canal to another part of the body or alternatively another region of the brain or spinal canal. Examples of shunts include, but are not limited to, ventriculoperitoneal (VP) shunts, ventriculoatrial (VA) shunts, or lumboperitoneal (LP) shunts among others (see also, e.g., United States Patent Application 20060020239, the contents of which are incorporated by reference).

“Signal” is used according to its art accepted meaning and refers to something that incites to action. In the context of the present invention, examples of signals include, but are not limited to, magnetostatic fields, magnetic fields, magnetic forces, electromagnetic fields, or electromagnetic forces. The magnetic field can be produced by the motion of electric charges, or electric current, and gives rise to the magnetic force associated with magnets. Magnetic fields or forces, for example, can be generated by a number of different means known in the art. Magnetic fields or forces can be generated by a magnet, typically a permanent magnet. Magnetic fields can also be generated by electromagnets, generally in the form of electrically conducting or superconducting coils. Moreover, batteries, microtransponders (see, e.g., U.S. Pat. No. 6,315,72 the contents of which are incorporated by reference), reluctance modulators (see, e.g., U.S. Pat. No. 3,240,051, the contents of which are incorporated my reference), or a rotating magnetic system can be used to generate a magnetic field alone or in combination with other elements.

The term “actuator” as used herein refers to a device that actuates, or moves or controls something via a movement to transfer motion from one object to another (e.g., via rotation, vibration, or linear movement). In typical embodiments, the actuator movement functions to transfer motion to a fluid in the fluid conduit. This physical movement of an actuator brushes cells and debris away from a catheter surface.

As used herein, “coating” as in a coating for inhibiting the accumulation of materials can comprise an antimicrobial agent and/or an anticoagulant agent that can be disposed on or incorporated into the fluid conduit material. Any antimicrobial agent, such as an antibacterial agent, an antiseptic agent, etc., can be used to prevent infection (see, e.g., United States Patent Application 20050008763 and U.S. Pat. No. 6,719,991, the contents of which are incorporated by reference). Moreover, it is also known in the art that photocatalytic agents can be incorporated into catheter materials (see also, e.g., U.S. Pat. No. 6,719,991, the contents of which are incorporated by reference).

The implantable medical device as disclosed by this invention can be used to treat a wide variety of other conditions in addition to hydrocephalus. Understandably, the actuator's physical dimensions will be adapted to integrate the devices into the medical device system of choice (e.g. round vs. square, different diameters, thickness, etc.). Additionally, mechanical, magnetic, and surface properties can be adapted to the parameters of each medical device system in order to achieve for example, optimal catheter clearing. Illustrative additional systems that can incorporate the invention include: Ommaya reservoir systems; external ventricular drainage systems; external lumbar CSF drainage systems; urinary bladder drainage systems; renal cyst catheters; pancreatic cyst catheters; ocular shunts (e.g. for glaucoma); inner ear shunts; liver bile shunts; pleural drainage catheters; pericardial drainage systems; lymphatic drainage systems and the like. Fluid conduits can also gain access to the CSF ventricular cisternal spaces, or cyst cavities, with the catheter terminating in a reservoir.

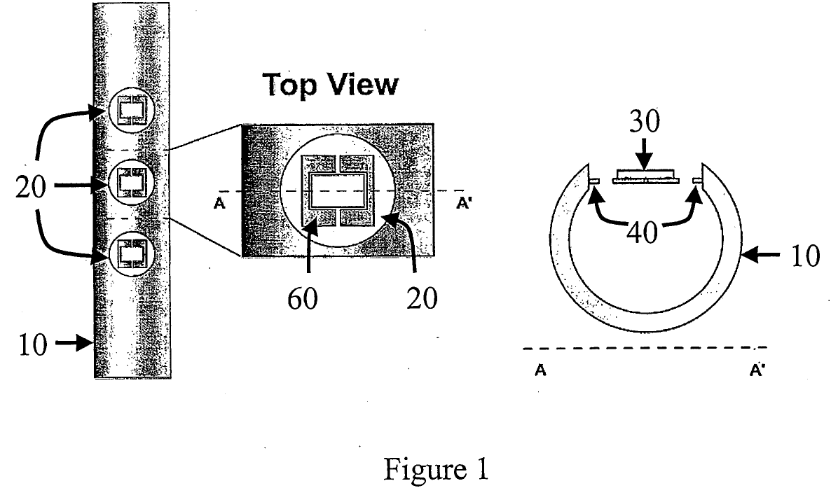

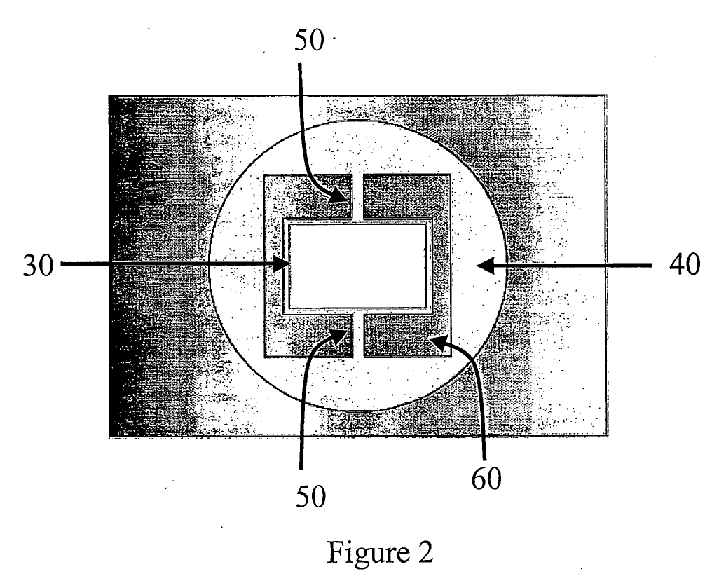

Referring now to the drawings in detail and initially to FIGS. 1 and 2 thereof, the presently preferred embodiment of the actuator, generally referred to by the numeral 20, comprises a moveable actuatable member 30 connected to a surrounding frame 40 by bridging element 50. The catheter device may be made up of one or more actuators 20.

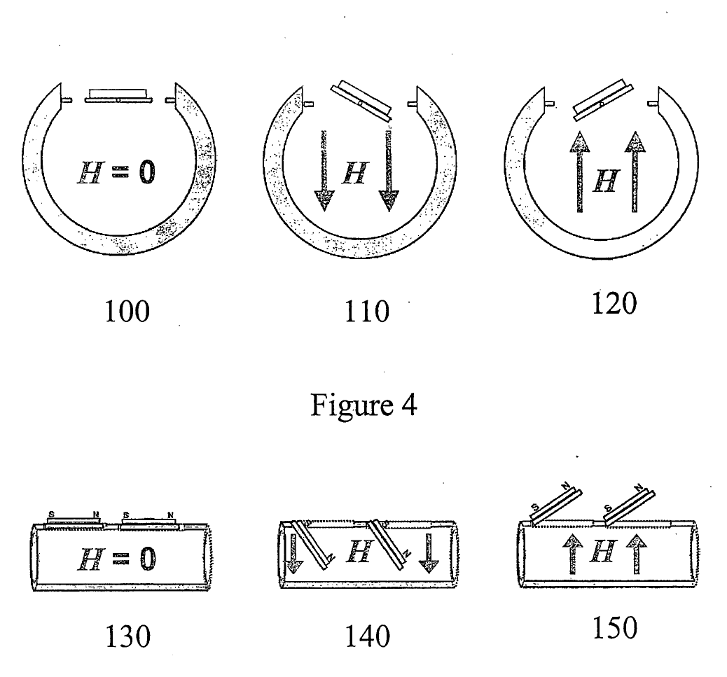

As shown in FIG. 4, the actuatable member 20 incorporates integral magnetic components that cause the moveable element to alternate between a first position 110 and a second position 120 in response to an external moving magnet field. The movement of the actuatable member will inhibit the formation of occluding materials at the orifice 60 of the fluid conduit 10. The movement of the actuatable member may also free attached cells, cellular debris, protein accumulation, and other biological process that may occlude the orifice of the fluid conduit. The actuatable member comprises an element that moves in response to a signal, alternating between a first and second position. This alternation can be, for example, but not limited to, a rotation, vibration, or linear movement that occurs e.g., inside, outside, on top of, or within the fluid conduit. The physical dimensions of the actuator/actuatable member can be adapted to integrate into the implantable medical device of choice. The actuatable member can also be constructed to have physical parameters that optimize its ability to alternate between a first and second position so as to inhibit accumulation of materials at an orifice of a fluid conduit. For example, the actuatable member can be constructed to have physical parameters that optimize its ability to sweep the orifice so as to inhibit accumulation of materials at the orifice of a fluid conduit. The actuatable member can, for example, have a particular structure that optimizes its performance, such as, but not limited to, an arm, a lever, a base, a disc, a flange, as well as the inclusion of additional simple mechanical means such as a pulley or a fulcrum or the like. An implantable medical device can have, for example, more than one type of actuatable member structure. Moreover, the actuatable member is typically comprised of a material that responds to a signal. For example, the actuatable member can be comprised of a material that responds to a magnetic signal or the actuatable member can be comprised of one or more materials that responds to an electromagnetic signal.

The implantable medical device as disclosed by this invention can be used to treat a wide variety of conditions such as hydrocephalus. Non-limiting examples of diseases that can be treated according to various embodiments of the invention include dementia, encephalopathy, encephalitis; meningitis; CNS infection and the like.

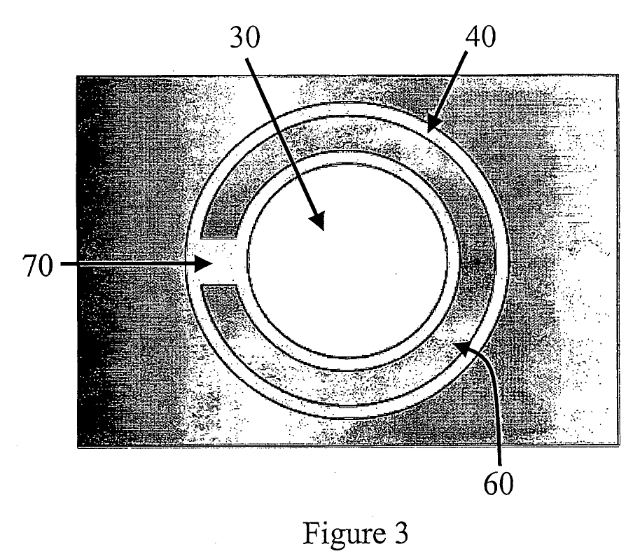

As shown in FIGS. 2 and 3, the bridging element 50 is the mechanical link between the moveable element 30 and the surrounding frame 40. The characteristics of the bridging element are 1) to provide an attachment point(s) of the moveable element 30 to the surrounding frame 40, 2) to return the moveable element 30 to its resting baseline position if not being deflected by the external magnet forces, 3) to allow sufficient movement of the moveable element 30 in order to fulfill the requirements of freeing or deterring biological/chemical occluding material, and 4) to prevent excessive movement of the moveable element 30 in its normal use or if submitted to large magnetic fields (such as an MRI). The moveable element 30 in the preferred configuration comprised in FIG. 4 act as a torsion element, although other configurations such as a cantilever in FIG. 2 may have also been manufactured and implemented.

The shown configuration of the surrounding frame 40 in FIGS. 2 and 3 is schematic in nature and the design not limited to what is shown. The design is such that a fluid conduit 10 is created between the surrounding frame 40 and the combination of the moveable element 30 and the bridging element 50 1) that allows movement of CSF and cellular elements within CSF through the fluid conduit 10 2) will be of optimal dimension so that movement of the moveable element will result in impediment and/or removal of biological/chemical occlusive material and 3) so that the combined channel area can be preset to a specified fluid flow resistance value. In embodiments of the invention, the fluid channel creates an “orifice”, which refers to a small hole or opening through which fluid flows. Examples of orifices described in the art include, but are not limited to, a pore, a flow hole, an inlet hole, or an outlet hole etc. As is known in the art, the orifice can be located at any appropriate location on the fluid conduit, e.g., on, within, outside, or above the fluid conduit. For example, an orifice can be provided in the proximal end portion of the ventricular catheter, providing fluid communication between the outer surface of the catheter and the catheter lumen. As is also known in the art, the orifice can be adapted for a specific purpose or in vivo environment, for example, to allow excess CSF present in the brain to drain into the shunt and onward into another location in the body. The orifice can be provided in the distal end portion of a drainage catheter, for example to provide fluid communication between the outer surface of a drainage catheter and a drainage catheter lumen. The orifice can be adapted to allow CSF within the shunt to drain into another portion of the patient's body, such as into the peritoneum.

A catheter device, shown as a possible configuration in FIG. 1, may be made up of one or more actuators 20. The actuators 20 may be separately integrated within the catheter material or manufactured in a neighboring configuration, as shown in FIG. 1, so that multiple actuators can coexist on a single surface, termed an actuator complex. The actuators 20 or actuator complexes can be situated at or near the terminus of the catheter, or anywhere along the path of the fluid conduit as single or multiple units and/or complexes.

The actuator devices can then be activated by an external magnetic field that guides the magnetic actuatable member to maintain a specific physical position. As the magnetic field is alternated, the actuatable member assumes a new position and in the process, mechanically sweeps the catheter surface and clears any cellular attachments. Frequent actuation within the catheter provides preventative treatment against occlusion due to cellular accumulation. Without an external magnetic force, the actuatable member sits passively within the catheter, allowing fluid to flow through the entire available area. By periodically sweeping the catheter surface, the device will remove any small cellular formations before the formation of a complete occlusion. A patient can activate the device in the privacy of their own home without compromising their daily activities. In one example of the embodiment, by placing the external magnetic field generator underneath their pillow while they sleep for example, the patient can rest comfortably while the actuatable member refreshes the ventricular catheter surface through the night. The invention, thereby, disclosed herein provides a number of advantages and improvements over existing technologies.

The actuators of the invention can be fabricated from a variety of different materials. In one embodiment of the invention the actuator is fabricated using a microelectromechanical (MEMS) technique. In embodiments of the invention, the actuator can be situated at the orifice of the fluid conduit and alternate between a first and a second position in response to the external signal or force. Optionally, the invention can comprise a MEMS device or MEMS actuatable elements. “MEMS devices” generally refer to devices on the micrometer size and which can include small 3D features of various geometries. They are typically manufactured using planar processing similar to semiconductor processes such as surface micromachining and/or bulk micromachining. These devices and elements generally range in size from a micrometer (a millionth of a meter) to a millimeter (thousandth of a meter). They are typically fabricated using modified silicon fabrication technology (used to make electronics), molding and plating, wet etching (KOH, TMAH) and dry etching (RIE and DRIE), electro discharge machining (EDM), and other technologies capable of manufacturing very small devices. Devices which use microfabrication methods can be made to include moving parts linked to electrical components for processes such as actuation. MEMS technologies are sometimes termed micromechanics, micro machines, or micro system technology (MST). The manufacture of the actuator, as described in the above embodiments, is not limited to MEMS techniques. Also, as disclosed herein, “MEMS actuatable elements” can include MEMS actuator arrays. The MEMS actuator arrays can be coated with a thin film of a material that improves the physical, chemical, or electronic, properties of the array, for example, including, but not limited to, polyimide. The MEMS actuator arrays can promote the sensorless manipulation of small objects and fluids (see, e.g., United States Patent Application 20060069425, the contents of which are incorporated by reference).

In one embodiment of the invention, the actuator and/or implantable medical device as disclosed herein can have one or more components that are highly compatible with MRI and other strong magnetic fields. Some exemplary materials include, but are not limited to, super-paramagnetic iron-oxide (SPIO) nanoparticles (see, e.g., United States Patent Application 20050261575, the contents of which are incorporated by reference); a radiopaque coating formed from a polymer loaded with a radiopaque material such as barium sulfate (see, e.g., U.S. Patent United States Patent Application 2005028304, the contents of which are incorporated by reference); an enhancing agent such as ferromagnetic particles within the polymeric material used to construct catheters (see, e.g., U.S. Pat. No. 5,154,179, the contents of which are incorporated by reference); a liquid or gel contrast agent containing a paramagnetic material into a catheter lumen (see, e.g., U.S. Pat. No. 5,154,179, the contents of which are incorporated by reference); paramagnetic ionic particles into non-metallic materials (see, e.g., U.S. Pat. No. 5,817,017, the contents of which are incorporated by reference); a hydrogel polymer coating (see, e.g., United States Patent Application 20030100830, the contents of which are incorporated by reference); a ceramic material that is thinly coated, by electroplating, sputtering or other deposition technique, etc., with a suitable electrode metal such as platinum, titanium, or alloys thereof (see, e.g., U.S. Pat. No. 6,968,236, the contents of which are incorporated by reference); or ferrofluids (see, e.g., U.S. Pat. No. 6,120,856, the contents of which are incorporated by reference). In addition, metal components can be used in the actuatable member that can be silver or silver-coated for enhanced antimicrobial resistance. Metal components used in the actuatable members can also be titanium and coated with titania. Plastic components used in the mechanical means can be optically transmissive plastics (see, e.g., U.S. Patent Application 20060004317, the contents of which are incorporated by reference).

The devices of the methods of the invention can include any of a wide variety of complementary elements known in the art. For example an actuator coupled to a fluid conduit can be disposed on a medical device having one or more cilia, flanges, slots, or other elements. The fluid conduit can have cilia that are movable as a group for moving particles. The fluid conduit can have flanges, which are thin-walled discs intended to physically shelter the drainage holes through the catheter wall. The fluid conduit can have slots. Slots are used to alleviate the bleeding caused when the tissue is torn by the proximal tip of the fluid conduit. If tissue grows into these slots and causes an obstruction to the flow of fluid therethrough, the tissue would not have to be torn to remove the fluid conduit, the fluid conduit tip simply slips out of the tissue.

### EXEMPLARY EMBODIMENTS OF THE INVENTION

The invention disclosed herein is designed for use with medical devices in which it is desirable to facilitate and/or modulate the flow of a fluid through a fluid conduit of the medical device. The invention disclosed herein has a number of embodiments including the following illustrative embodiments. Those of skill in the art will appreciate that the invention is not limited to these embodiments, and that these are merely illustrative examples of the wide variety of ways in which the instant invention can be used with medical devices.

One illustrative embodiment is an implantable medical device comprising a fluid conduit having an orifice; and an actuator coupled to the orifice of the fluid conduit; wherein the actuator alternates between a first and second position in response to a signal so as to inhibit the accumulation of materials and/or prevent the obstruction at the orifice of the fluid conduit. Such embodiments of the invention are adapted to facilitate fluid flow through the fluid conduit of a medical device by inhibiting or decreasing the build up of materials that can occlude an orifice of a fluid conduit. Illustrative embodiments of the invention include devices having an actuator coupled to a fluid conduit such as a catheter of a medical device. Optionally, this actuator is fabricated using micro-electro-mechanical system (MEMS) technology. Embodiments of the invention are designed to prevent and/or reverse the occlusion in fluid conduits in implantable medical devices, for example by periodically sweeping a catheter orifice surface using a magnetic actuator.

Other embodiments of the invention are adapted to facilitate and/or modulate the flow of fluids from one region of the body to another and can be used for example with those medical devices that are designed to facilitate the drainage of cardiac fluids, ascites fluids, ocular fluids, brain fluids, urinary fluids, synovial fluids, hepatic fluids and the like. Other embodiments of the invention are adapted to facilitate and/or modulate the flow of a fluid medication such as a fluid medication that is dispensed from a medical device such as a medication delivery pump (e.g. an insulin pump). One of skill in the art understands that the above-noted devices are merely illustrative and that embodiments of the invention can be used with any medical device having a fluid conduit prone to occlusion.

Another illustrative embodiment is an implantable medical device comprising a fluid conduit having an orifice; and an actuator coupled to the orifice of the fluid conduit; wherein the actuator alternates between a first and second position in response to a signal so as to inhibit accumulation of materials at the orifice of the fluid conduit. In some embodiments of the invention, the fluid conduit is adapted for use as a shunt. In a highly preferred embodiment of the invention, the shunt is adapted for use in the treatment of hydrocephalus. Typically, the fluid conduit having an orifice is a catheter. In some embodiments of the invention, the actuator is adapted to sweep a particular material accumulated at the orifice of the fluid conduit such as red blood cells, calcium deposits, growing tissue, cellular debris, or inflammatory cells. Optionally, the implantable medical device further comprises a coating composition that inhibits the accumulation of materials at the orifice of the fluid conduit. In some embodiments, the composition of the actuator is selected to be compatible with MRI. Typically, the signal is a remote signal. In some embodiments, the signal comprises a magnetic signal. In a highly preferred embodiment of the invention, the magnetic signal comprises an external AC magnetic field. Typically, the actuator proximal to the orifice of the fluid conduit alternates in a sweeping motion. In some embodiments of the invention, the actuator alternates between the first and the second position in response to the signal so as to modulate fluid flow through the fluid conduit.

In a specific illustrative embodiment of the invention, custom-fit MEMS fabricated magnetic actuators are constructed to allow their easy integration into existing implantable medical devices. In one such embodiment, the devices are activated by an external magnetic field that guides the magnetic actuators to maintain specific physical positions. Without an external magnetic force, the magnetic actuators sits passively within the catheter, allowing fluid to flow through the entire available area. As the magnetic field is alternated, the magnetic actuators can assume a new position and in the process, mechanically sweep the catheter surface and clear any potentially occluding materials such as cellular attachments. By periodically sweeping the catheter surface, the device removes any accumulations of cells and proteins etc. before the formation of a complete occlusion. The magnetic actuator's movement is not limited to sweeping motions and can comprise a wide variety of movements. Frequent actuation within the catheter can in this way prevent and/or reduce the buildup of occlusions such as those that occur due to cellular accumulation.

Another embodiment of the invention is a method of inhibiting formation of occluding materials at an orifice of a fluid conduit of an implanted medical device, the method comprising using a signal to actuate an actuator disposed at the orifice of the fluid conduit; wherein the actuator alternates between a first and a second position in response to the signal and inhibits the formation of occluding materials at the orifice of the fluid conduit. In an illustrative embodiment, the fluid conduit having an orifice is adapted for use as a shunt. In a specific embodiment of the invention, the shunt is adapted for use in the treatment of hydrocephalus. Typically, the signal comprises an external magnetic signal. In some embodiments, the method of inhibiting formation of occluding materials at the orifice of the fluid conduit could further comprise coating the fluid conduit with a composition that inhibits the accumulation of materials at the orifice of the fluid conduit. Typically, the actuator alternates between the first and the second position in a manner comprising a sweeping, vibrating, or rotating motion. In some embodiments of the invention, a device that generates the signal is placed in proximity to the head of the patient.

Another embodiment of the invention is a method of decreasing the amount of materials accumulated at an orifice of a fluid conduit of an implantable medical device, the method comprising, using an external signal to actuate a actuatable member disposed at the orifice of the fluid conduit; wherein the actuatable member alternates between a first and a second position in response to the external signal so that the amount of materials accumulated at the orifice of the fluid conduit is decreased.

One illustrative embodiment of the invention utilizes actuators fabricated via micro-electro-mechanical system (MEMS) technologies. The actuators of the invention can also be fabricated by any of the variety of other well known manufacturing processes known in the art. In designing any specific embodiment of the invention, one can simply fabricate one or more devices and test them using biological tissues to determine the optimal force that will clear an obstruction in a lumen. Understandably, in designing the devices, artisan will select (and/or determine) biocompatible materials that will be safe for use over an extended period of time.

In an illustrative embodiment of the invention, custom-fit magnetic actuators (e.g. MEMS manufactured magnetic actuators) can be integrated into the orifice that allow fluid to flow through a catheter. The devices can then be activated by an external magnetic field that guides the magnetic actuatable member to maintain a specific physical position. As the magnetic field is alternated, the actuatable member assumes a new position and in the process, mechanically sweeps the catheter surface and clears any cellular attachments. Frequent actuation within the catheter provides preventative treatment against occlusion due to cellular accumulation. Without an external magnetic force, the actuatable member sits passively within the catheter, allowing fluid to flow through the entire available area. By periodically sweeping the catheter surface, the device will remove any small cellular formations before the formation of a complete occlusion. A patient can activate the device in the privacy of their own home without compromising their daily activities. By placing the external magnetic field generator underneath their pillow while they sleep for example, the patient can rest comfortably while the actuatable member refreshes the ventricular catheter surface through the night. The invention, thereby, disclosed herein provides a number of advantages and improvements over existing technologies.

Currently all implanted (temporary or permanent) catheters that access bodily fluids (including but not limited to cerebrospinal fluid, aqueous humor, urine, blood) are prone to obstruction at the catheter inlet. Embodiments of the invention provide an improved implantable catheter with incorporated actuators with the application of movement and/or drainage of bodily fluids in (or fluids infused into) a patient. This embodiment of the invention provides a novel approach for clearing or preventing occlusion of implantable catheters, the general class of composition-of-matter (materials) used to enable the magnetomechanical system, and the fabrication of the device. Embodiments of the invention can be used in a wide variety of contexts, with the treatment of hydrocephalus (cerebrospinal fluid drainage) provided as one illustrative embodiment. Some embodiments of the invention are situated at the proximal catheter from where the bodily fluid is drawn. In typical embodiments of the invention, the entry or exits ports of the catheter (e.g. for the drainage of bodily fluids), contains the actuators. For the treatment of neurosurgical disorders (for example hydrocephalus), the catheter can be one part of a CSF-shunt system (which typically includes a ventricular catheter, a valve, and a distal catheter). For other applications (e.g. indwelling bladder catheters, ocular shunts for glaucoma, venous catheters, etc.), the anti-occlusion component can be incorporated into existing catheter setups (with modification of the proximal catheter design and manufacture to accommodate our invention). The MEMS actuatable member will typically require an external magnetic field generator to actuate the devices. Those of skill in the art understand that while external activation of the actuators disclosed herein is preferred in certain contexts, the invention is not limited to such embodiments. One can for example power actuators through the catheter with wires and locally generated magnetic fields.

Another embodiment is an implantable medical device comprising a fluid conduit having an orifice; and an actuator coupled to the orifice of the fluid conduit, wherein the actuator alternates between a first and a second position in response to a signal so as to inhibit accumulation of materials at the orifice of the fluid conduit. A specific embodiment of the invention is an CSF shunt comprising a fluid conduit having an orifice; and a magnetic actuatable member proximal to the orifice of the fluid conduit wherein the actuatable member alternates between a first and a second position in response to a magnetic signal so as to inhibit accumulation of materials at the orifice of the fluid conduit.

Yet another embodiment of the invention is a method of inhibiting formation of occluding materials at an orifice of a fluid conduit of an implanted medical device comprising: using a signal to actuate an actuator disposed at the orifice of the fluid conduit; wherein the actuator alternates between a first and a second position in response to the signal and inhibits formation of occluding materials at the orifice of the fluid conduit. A related embodiment is a method of decreasing the amount of materials accumulated at an orifice of a fluid conduit of an implanted medical device comprising: using an external signal to actuate an actuatable member disposed at the orifice of the fluid conduit; wherein the actuatable member alternates between a first and a second position in response to the external signal so that the amount of materials accumulated at the orifice of the fluid conduit is decreased.

Another illustrative embodiment is an implantable medical device having a fluid conduit comprising an actuatable member that can assume a first and a second position within a fluid conduit of the medical device, wherein the actuatable member can be controlled by a user to alternate between the first and the second position. Typically, the actuatable member can be externally controlled. In some embodiments of the invention, the actuatable member alternates between the first and the second position in a manner that inhibits the formation of materials on a surface of a fluid conduit (e.g. a catheter). In other embodiments, the actuatable member decreases the amount of material accumulated at the orifice of a fluid conduit. In other embodiments of the invention, the actuatable member alternates between the first open and a second closed position in a manner that controls fluid flow through the conduit. Optionally in such devices, the actuatable member is a magnetic actuatable member fabricated using a micro-electro-mechanical system technology.

Another embodiment of the invention is a method of modulating fluid flow through a fluid conduit of a medical device, wherein the fluid conduit comprises a actuatable member that can assume a first and a second position within the fluid conduit of the medical device, the method comprising alternating the position of the actuatable member between the first and the second position in a manner that modulates fluid flow through the fluid conduit of the medical device. In an illustrative embodiment, alternating the position of the actuatable member between the first and the second position inhibits the formation of materials on a surface of the fluid conduit so that fluid flow through the fluid conduit is facilitated. In a specific embodiment of the invention, the medical device is a catheter used in the treatment of hydrocephalus.

The basic actuator design disclosed herein can be adapted for use with a variety of medical devices in which it is desirable to facilitate and/or modulate the flow of a fluid. For example, some specific embodiments of the invention are adapted to facilitate fluid flow through the fluid conduit of a medical device such as a catheter. Other embodiments of the invention are adapted to facilitate the flow of fluids from one region to another such as the drainage of cardiac fluids, ascites fluids, ocular fluids, brain fluids, urinary fluids, synovial fluids, hepatic fluids and the like. Other embodiments of the actuatable member design are adapted to modulate the flow of a fluid medication such as a fluid medication that is dispensed via tubing from a medication delivery pump (e.g. an insulin pump). Moreover, embodiments of this invention can be integrated into a variety of medical devices and do not need to be modified to perform either fluid drainage and/or medication delivery.

Understandably, the actuator's physical dimensions will be adapted to integrate the devices into the medical device system of choice (e.g. round vs. square, different diameters, thickness, etc.). Additionally, mechanical, magnetic, and surface properties can be adapted to the parameters of each medical device system in order to achieve for example, optimal catheter clearing. Illustrative additional systems that can incorporate the invention include: Ommaya reservoir systems; external ventricular drainage systems; external lumbar CSF drainage systems; urinary bladder drainage systems; renal cyst catheters; pancreatic cyst catheters; ocular shunts (e.g. for glaucoma); inner ear shunts; liver bile shunts; pleural drainage catheters; pericardial drainage systems; lymphatic drainage systems and the like.

Separate aspects of the actuatable member design can be modified independently of each other to optimize actuatable member performance. A first aspect of actuatable member design is structural. In this context, by changing either the actuator's dimensions or structural materials or both can alter the mechanical properties of the actuator. For applications that require more torque to remove occlusions, the device can be fabricated with a larger volume of magnetic element(s). In order to produce a more robust structure, one can also increase the width and/or thickness of the torsion beams or use a different type of mechanical support, such as a cantilever beam. Similarly, the volume of the magnetic element(s) can be reduced for applications requiting less force and the mechanical support can be made less stiff. Understandably, the device (e.g. catheter) dimensions will provide the parameters for the actuatable member dimensions. As for structural materials, virtually any nonferrous material can be used to fabricate the mechanically supporting structural material. The force and flexibility required for occlusion removal will determine the type of mechanical material properties needed (i.e., more flexible materials, such as organic polymers, or more rigid materials, such as inorganic crystalline or polycrystalline films, such as single-crystal silicon, polycrystalline silicon, silicon nitride, silicon carbide, etc.). One type of material that can be manipulated are the magnetic materials. For applications that require more torque to remove occlusions, the device can be fabricated with materials that have a higher remnant and saturation magnetization and coercivity. Another type of material that can be manipulated are the interface materials. Virtually any nonferrous material can be used to coat the device. Biocompatibility issues will guide the materials used to coat the device, including any surface modifications. A factor in determining the biocompatibility of the device will be the biological environment in which the device will be functioning. Additionally, polyethylene glycol or any other surface modification can be immobilized in a material coated on the actuatable member surface to discourage short-term protein adhesion and cell growth.

The actuators disclosed herein can be manufactured according to well known technologies. A preferred manufacturing technique/process used to construct the actuatable member of the invention is micro-electro-mechanical system (MEMS) process. MEMS allow the manufacture of actuators at a size scale comparable to individual cells or groups of cells. This will provide a mechanical leverage advantage. Second, MEMS allows economy of scale with regards to the mass manufacture of catheters (which provides a marketing/sales advantage). However, we are not limiting the manufacturing process to MEMS techniques. The approach of using a magnetic MEMS actuatable member can be accomplished via other manufacturing techniques.

In typical embodiments of the invention, the magnetic field generator is remote, that is not physically connected to the shunt system. The magnetic field generator provides the field necessary for the actuators to function. The catheter holds the actuatable member in the proper position (e.g. in orifice). The catheter is not specialized for the device and almost any commercial catheter can be used to integrate the device. Similarly, alternate valve or distal catheter designs can be used in the shunt system. Any device that can produce an alternating magnetic field can be used to actuate the device. For the catheter and field generator, modifications can be made to customize the component for function with the actuator.

The actuators can be fabricated with a variety of different materials (i.e. different structural or magnetic materials). Additionally, the catheter can be designed to include a modified surface to reduce obstruction. The actuators can be fabricated from various different materials to generate larger or smaller forces to target obstructions occurring in other biological catheters. Additionally, the scale of the devices can be customized to fit different catheter dimensions. Lastly, surface coatings can be added over the catheter to provide additional short-term occlusion resistance. Though an external magnetic field can be generated from existing devices, a specialized device to produce a magnetic field customized for actuatable member function can produce the most effective catheter-clearing properties. The field parameters (amplitude, frequency, wave signal, etc) would be specialized for the actuator. In theory, a MRI scanner can be used to activate the device.

Technologies used to produce embodiments of the invention are well known in the art. Micromachining processes for example have long been used to produce solid-state integrated circuits with features ranging from millimeters to micrometers and now nanometers (see e.g., Wolf S et al., (2000) Silicon Processing for the VLSI era Vol 1. Los Angeles: Lattice Press). In the 1980s it became clear that micromachining processes can also be used to produce mechanical elements, such cantilevers and bridges, again with dimensions on the scale of micrometers. Since then, there has been an explosion of research and commercialization efforts seeking to develop complex integrated micro-electro-mechanical systems (MEMS), such as microsensors and actuators. When appropriate, integrated circuits are also integrated with MEMS to realize complete microsystems. Initially the leading examples of MEMS devices were inertial sensors (e.g., accelerometers, gyros, etc.) and pressure sensors (see e.g., Data Sheet NPC-107 Series Disposable Medical Pressure Sensor, Lucas NovaSensor, 1055 Mission Court, Fremont, CA 94539). Now, however, a truly wide range of devices is being developed and commercialized for many different markets: optical MEMS (optical switches, displays, and scanners); BioMEMS (microfluidics, cell manipulation, gene chips), RF MEMS (switches and filters for next-generation chip-scale communication systems) (see e.g., G. T. A. Kovacs, Micromachined Transducers Sourcebook. WCB/McGraw-Hill, 1998, ISBN 0 07-290722-3). Since many applications have restrictions that limit the range of material used, micromachining techniques have been successfully developed for many different material sets (e.g., not only the conventional rigid silicon, oxides, and metals, but now also glasses, flexible plastics and polymers, and even biological compounds and tissues). One of skill in the art can readily determine the most appropriate materials to ensure both biocompatibility and MRI-compatibility.

One of the key reasons for using micromachining technologies is their tremendous ability to produce large quantities of identical precision-machined parts with only minimal added expense. Thus micromachining is a batch-fabrication process that can take advantage of economies of scale to produce many inexpensive components. The MEMS devices of the invention can for example be batch-fabricated to produce many actuators with each fabrication process. As a result, the device can be produced quickly and at a low cost. Additionally, our design employs physical components to clear the device and produces improved performance over existing designs. By producing a self-clearing MEMS catheter, we will greatly decrease the probability of shunt obstruction. In turn, patients will require fewer replacement surgeries, face less risk and less stress associated with surgeries and spend more time enjoying the benefits of a properly functioning shunt.

Micromachining has been used to realize actuators driven by a variety of forces (e.g., electrostatic, piezoelectric, thermal, magnetostatic, etc.) (see e.g., G. T. A. Kovacs, Micromachined Transducers Sourcebook. WCB/McGraw-Hill, 1998, ISBN 0 07-290722-3). The following are examples of actuators driven by these transduction mechanisms: electrostatic digital mirror display (DMD) developed by Texas Instruments and used today in conference-room and movie-theater projectors (see e.g. Van Kessel P F et al., (1998) Proc. IEEE, (86): 1687-704), piezoelectric inch-worm stepper motors (see e.g., Judy J W et al., IEEE Transaction on Ultrasonics, Ferroelectrics and Frequency Control, vol. UFFC-37, no. 5, September 1990, pp. 428-437), thermally driven microfluidic valves (see e.g., Barth P W et al., Technical Digest Solid-State Sensor and MEMS actuatable member Workshop, 1994, pp. 248-250), and magnetically driven optical cross-connect switches (see e.g., Judy J W et al., IEEE Journal of Microelectromechanical Systems, vol. 6, no. 3, September 1997, pp. 249-256). Each method of actuation mentioned above has its own unique set of advantages and disadvantages.

Table 1 below provides a summary of certain advantages and disadvantages for the primary methods of actuation, we see that magnetostatic actuation appears to be optimal for use with an implanted hydrocephalus catheter. The key advantages of magnetostatic actuation are that it does not requite direct electrical connections, can operate in a conductive fluid environment, and can be driven by a remote source of magnetic field. The ability to use a remote source of magnetic field to control the movement of the implanted magnetic actuators eliminates concerns about electrical-lead failure and the incorporation and maintenance of an implanted power supply.

### Physiological Processes Pertinent to the Invention

An understanding of the wide variety of embodiments of the invention is facilitated by an understanding of the physiological processes pertinent to the invention. As is known in the art, there are a number of purported causes of shunt obstruction. Certain factors contributing to the obstruction of fluid conduits (e.g., ventricular catheter obstructions) include ventricular collapse due to excessive drainage, choroid plexus tissue migration and ingrowth, and gradual accumulation of cells in flow holes. In this context, an initial step in the design of self-clearing implantable devices (such as an effective self-clearing ventricular catheter) is an understanding of the processes that lead to occlusions. By analyzing the cellular composition of the catheter occlusions for example, one can begin to understand the mechanism and cells involved in obstruction formation and consider ways to address these issues.

Research describing the mechanisms and the cell types involved in the accumulation of cells and other materials in the flow holes has been limited. Presently, the problem is not well understood and no advances have been made that properly address this concern. Even though CSF normally has few cells, over the life of the shunt millions of cells will traverse it. Hydrocephalus conditions associated with pleocytosis, such as chronic meningitides, are known to shorten shunt half-lives. Addressing the issue of gradual cell accumulation can provide one of the greatest benefits in solving the problem of ventricular-catheter obstruction.

Pathological studies have shown that the cellular composition of catheter obstructions consists mainly of calcium formation, red blood cells, ependymal cells, and inflammatory tissue (see e.g., Brydon, H L et al., (1996), Neurosurgery, 38(3):498-504; Del Bigio M. R., (1998), Neurosurgery, 42(2):319-25; Echizenya, K. et al., (1987), J Neurosurg., 67(4):584-91; Gower D J et al., (1984) J Neurosurg., 61(6):1079-84; Koga H et al., (1992), Neurol Med Chir (Tokyo). 52(11):824-8; Lazareff J A et al., (1998), Childs Nerv. Syst., 14(6):271-5; Schoener W F et al., (1991) pp. 452-72 in S. Matsumoto, N. Tamaki (eds.) Hydrocephalus. Pathogenesis and Treatment. New York: Springer-Verlag; Snow R B et al., (1989) Surg. Neurol., 31(3):209-14; and Ventureyra, E. C. et al., (1994). Neurosurgery, 34(5):924-6, the content so which are incorporated by reference).

Ventricular collapse following shunting procedures is also clearly associated with shunt obstruction. In this context, a primary focus of design improvement has been to limit excessive drainage and therefore prevent the collapse of the ventricles. Although the incorporation of valves with an adjustable opening differential pressure that control the rate of CSF flow have been touted to maintain an ideal ventricular size and intracranial pressure, these goals have not been consistently achieved clinically. The mechanism by which obstruction occurs with ventricular collapse is clearly related to the direct apposition of ependymal and/or choroid plexus tissue with the ventricular catheter tip. As a result, cellular obstruction of the ventricular catheter flow holes occurs (see e.g., Drake, J. M., (1995) The Shunt Book. Cambridge: Blackwell Science). Despite advances in valve technologies, ventricular collapse continues to be an risk in shunt obstruction.

Choroid plexus tissue migration also occurs in situations where the catheter flow holes are in close proximity with the choroid plexus. The suctioning effect inherent with many shunt designs, can draw the choroid tissue directly into the catheter holes. Flanged catheter tips were introduced many years ago with the goal of preventing the choroid tissue from accessing the flow holes. The clinical experience with this design, however, has been mixed. Proximal catheter obstructions have not been prevented and the reason is not clear. Assuming choroid tissue was indeed impeded, cells freely floating in CSF presumably led to the obstruction. Some studies have suggested that optimal placement of the catheter tip is at a location that is out of the reach of the choroid plexus. Anatomically, this placement goal is very difficult to achieve with current catheter designs. With better catheter designs and judicious use of endoscopy, this placement goal can be achieved.

Though not commonly present in CSF, red blood cells may be introduced in situations of hemorrhaging either prior to or following shunt placement (see e.g., Brydon, HL et al., (1996), Neurosurgery, 38(3):498-504; Del Bigio M. R., (1998), Neurosurgery, 42(2):319-25). Highly susceptible to coagulating, red blood cells may form larger masses that easily obstruct catheters. Proper patient selection and good surgical techniques can reduce this risk, but not eliminate it.

Ependymal-tissue ingrowth can result from a catheter being placed too close to the ventricle wall. Similar to choroid plexus ingrowth, this type of occlusion is not formed by gradual cell accumulation and may be resolved by modifying the protocol for catheter placement, or the development of a catheter with modified rigidity to restrict the catheter from resting against the ventricle wall. It is probable that hydrocephalic patients may contain a higher amount of free-floating ependymal cell debris in their CSF. As the ventricle expands with increasing CSF accumulation, ependymal cells compensate by expanding and flattening. Soon after this change in cellular conformation, the cells lining the ventricles slough off and create an increased concentration of ependymal cell debris (see e.g., Bruni J E et al., (1985) Brain Res., 356(1):1-19). As dead-cell debris, ependymal cells are unlikely to be the cause of cell accumulation, but rather contributors to a previously formed cell mass. In ventricular catheter obstructions (see e.g., Lazareff J A et al., (1998), Childs Nerv. Syst, 14(6):271-5 and Ventureyra, E. C. et al., (1994). Neurosurgery, 34(5):924-6) ependymal cells have two possible forms. Inflammatory cell types are frequently identified in the literature as a main cause of occlusion due to cell accumulation (see, e.g., Del Bigio M. R., (1998), Neurosurgery, 42(2):319-25; Gower D J et al., (1984) J Neurosurg., 61(6):1079-84; Koga H et al., (1992), Neurol Med Chir (Tokyo). 32(11):824-8; Lazareff J A et al., (1998), Childs Nerv. Syst., 14(6):271-5; Schoener W F et al., (1991) pp. 452-72 in S. Matsumoto, N. Tamaki (eds.) Hydrocephalus. Pathogenesis and Treatment New York: Springer-Verlag; Snow R B et al., (1989) Surg. Neurol., 31(3):209-14; and Kossovsky N. et al., (1989) J Biomed Mater Res., 23(A1 Suppl):73-86). Though leukocytes are present in relatively small concentrations in the CSF, these cells are active and capable of sophisticated hypersensitivity reactions, including cell adhesion (see e.g., von Recum AF et al., (1995) J Biomater. Sci. Polym. Ed., 7(2):181-98). Almost all the white-blood-cell types are mentioned: lymphocytes, neutrophils, monocytes, and eosinophils. In addition, activated monocytes such as macrophages and multi-nucleate giant cells have also been reported to contribute cells.

Of all the aforementioned sources, the most probable sources of cell accumulation leading to catheter obstruction are inflammatory cells. It has been hypothesized that varying rates of catheter occlusion due to cell accumulation may result from a variation in the delayed hypersensitivity responses (see e.g., Gower D J et al., (1984) J Neurosurg., 61(6):1079-84; Snow R B et al., (1989) Surg. Neurol., 31(3):209-14; and Kossovsky N. et al., (1989) J Biomed Mater Res., 23(A1 Suppl):73-86). Although this mechanism of cell accumulation on silicone implants in the body has been well documented by pathologists (see e.g., von Recum A F et al., (1995) J Biomater. Sci. Polym. Ed., 7(2):181-98 and Takemoto Y et al., (1989) ASAIO Trans., 35(3):354-6) little research has focused on the cellular response to neurosurgical implants. It is unclear whether the mechanisms of white-blood-cell activation in the body have any similarities to those present in the ventricles.

Calcium formation has been listed in earlier studies as a significant cause of proximal catheter obstruction. (see e.g., Echizenya, K. et al., (1987), J Neurosurg., 67(4):584-91). Barium, a radiopaque material used to mark the catheters, was the target of calcium formation (replacement). Newer catheter designs that shield the barium from contact with the body have greatly eliminated this phenomenon.

As in implanted devices, surface chemistry and structure are typically critical in determining shunt biocompatibility. Cells from the host initially interact with only the surface; therefore, the surface properties of the implant determine the body's response to the entire implant (see e.g., Takemoto Y et al., (1989) ASAIO Trans., 35(3):354-6 and Ikada, Y., (1994). Biomaterials, 15 (10): 725-736). One known similarity between silicone implants in the body and ventricular catheters implanted in the brain is the formation of a protein coating over the silicone (see e.g., Gower D J et al., (1984) J Neurosurg., 61(6):1079-84 and Kossovsky N. et al., (1989) J Biomed Mater Res., 23(A1 Suppl):73-86). Immediately following implantation into the body, proteins from body fluids adsorb onto the implant surface. The implant surface is now biological in nature and can interact with the body in purely biological reactions (see e.g., Takemoto Y et al., (1989) ASAIO Trans., 35(3):354-6). Although the protein concentration in CSF alone does not contribute to catheter obstruction (see e.g., Brydon, HL et al., (1996), Neurosurgery, 38(3):498-504), its interactions with inflammatory cells may initiate reactions that lead to adhesion. In order to fully understand the steps that lead to cellular occlusion within the ventricle, one must first understand protein-implant adhesion to, then protein-cell interaction.

The main factors that influence protein adhesion in blood-contacting implants have been researched extensively and are found to be protein concentration and transport rate, surface hydrophobicity, protein stability, and surface charge (see e.g., Ikada, Y., (1994). Biomaterials, 15 (10): 725-736; Ratner, B D et al., (1996) Biomaterials Science. San Diego:Academic Press and Elbert, D L et al., (1996) Annu. Rev. Mater. Sci, 26: 365-94). The significance of protein concentration and transport rate simply describes the number of proteins that are physically present at the substrate surface and available for adhesion. CSF, consisting of up to 40% protein, continuously flows through and around the silicone catheter bringing the proteins in close proximity to the catheter surface. Aside from the physical presence of the protein at the surface, hydrophobicity is the dominating force that determines whether a protein adsorbs (see e.g., Elbert, D L et al., (1996) Annu. Rev. Mater. Sci, 26: 365-94). Catheters such as ventricular catheters are typically constructed with hydrophobic polymer materials such as silicone, a polymer composed of an inorganic siloxane backbone with pendant organic groups. Proteins with hydrophobic amino acid residues can easily displace the weak hydrogen bonds between water and a hydrophobic surface, thus lowering the overall surface energy. After contacting the surface, the protein may change confirmation to create a new lowered energy (see e.g., Elbert, D L et al., (1996) Annu. Rev. Mater. Sci., 26: 365-94). Assuming the proteins in solution are shielding their hydrophobic amino-acid side chains by folding them inward, a hydrophobic surface may also cause the protein to denature and expose the side-chains, irreversibly binding to the surface (see e.g., Ratner, BD et al., (1996) Biomaterials Science. San Diego:Academic Press and Elbert, D L et al., (1996) Annu. Rev. Mater. Sci., 26: 365-94). Additionally, charged surfaces are more likely to adsorb proteins of opposite charge in an attempt to resolve the excess surface charge and lower surface energy. Adding to the complexity, proteins that originally adsorb onto a surface may be displaced by others that are capable of bonding more strongly to the surface. Thus, the implant surface is dynamic and the protein layer becomes more strongly adhered as time progresses.

After the formation of a protein layer, the implant displays a biological surface to the body that is able to undergo biological reactions with host cells (see e.g., Ratner, B D et al., (1996) Biomaterials Science. San Diego:Academic Press; Elbert, D L et al., (1996) Annu. Rev. Mater. Sci., 26: 365-94; and Norde, W., (1995) Cells and Materials, 5 (1): 97-112). Biological recognition, mediated by protein interactions with cell surfaces, leads to cell adhesion. The integrin receptor class mediates both cell-surface and cell-cell interactions by reacting with protein ligands at relatively small, localized sites. Integrin-binding sequences tend to be variations of RGD sequences, with different integrins binding specifically to certain protein ligands (see e.g., Norde, W., (1995) Cells and Materials, 5 (1): 97-112). The adsorbed protein has many sites and can bind to the integrin as well as other receptors. Pathological studies of silicone implants in the body have described leukocyte adhesion to surface proteins due to arginine-glycine-glutamic acid (RGD) receptor-ligand binding (see e.g., Takemoto Y et al., (1989) ASAIO Trans., 35(3):354-6). Though CSF lacks red blood cells and other cell types present in whole blood, similarities may exist between the general mechanisms in which cells and proteins adhere on implants.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. Throughout this application, various publications are referenced. The disclosures of these publications are hereby incorporated by reference herein in their entireties.

TABLE 1

| Method of | | |
|---|---|---|
| Microactuation | Advantages | Disadvantages |
| Electrostatic | easy to integrate with micromachining | cannot operate in a conductive |
| | technologies | fluidic environment |
| | generates significant forces when the | requires direct electrical |
| | gap between electrodes is small | connection |
| | (<10 μm) | |
| | consumes very little power | |
| Piezoelectric | generates very large forces | range of movement is extremely |
| | can operate over a wide range of | limited (<1 μm) |
| | frequencies | requires direct electrical |
| | stepper motor configurations that | connection |
| | make use of rapid, small, and powerful | often requires very large |
| | steps are effective | voltages (>100 V) |
| Thermal | easy to integrate with micromachining | requires direct electrical |
| | generates significant forces | connection |
| | | consumes a lot of power |
| | | heat from actuator may be |
| | | problematic for implants |
| Magnetostatic | relatively easy to integrate with | ferromagnetic materials (e.g., |
| | micromachining | Ni, Fe, Co) must be coated with |
| | generates significant forces with a | a corrosion-resistant layer to |
| | strong field | prevent dissolution and the |
| | does not require any direct electrical or | associated contamination |
| | mechanical connections (i.e., | |
| | microactuators can be operated by a | |
| | remote source for the magnetic field) | |
| | zero device-level power consumption | |
| | (energy is consumed by the remote | |
| | source for the field) | |

What is claimed is:

1. An implantable medical device comprising:
a fluid conduit having an orifice; and
an actuator coupled to the orifice of the fluid conduit,
wherein the actuator alternates between a first and a second position in response to a signal so as to inhibit accumulation of materials or remove materials at the orifice of the fluid conduit.

2. The implantable medical device of claim 1, wherein the fluid conduit is adapted for use as a shunt.

3. The implantable medical device of claim 2, wherein the shunt is adapted for use in the treatment of hydrocephalus.

4. The implantable medical device of claim 1, wherein the fluid conduit having an orifice comprises a catheter.

5. The implantable medical device of claim 1, further comprising a coating composition that inhibits the accumulation of materials at the orifice of the fluid conduit.

6. The implantable medical device of claim 1, wherein a composition of the actuator is selected to be compatible with strong magnetic fields such as MRI.

7. The implantable medical device of claim 1, wherein the signal is a remote signal.

8. The implantable medical device of claim 1, wherein the signal comprises a magnetic signal.

9. The implantable medical device of claim 8, wherein the magnetic signal comprises an external oscillating magnetic field.

10. The implantable medical device of claim 1, wherein the actuatable member at the orifice of the fluid conduit moves in such a manner to prevent or reduce occlusion of the orifice of the fluid conduit.

11. The implantable medical device of claim 1, wherein the actuator restricts fluid flow through the fluid conduit.

12. A method of removing materials or decreasing materials that accumulate at an orifice of a fluid conduit of an implantable medical device comprising
using an external signal to actuate an actuatable member disposed at the orifice of the fluid conduit;
wherein the actuatable member alternates between a first and a second position in response to the external signal so that the materials are removed or the accumulation of materials at the orifice of the fluid conduit is decreased.

13. The method of claim 12, wherein the fluid conduit having an orifice is adapted for use as a shunt.

14. The method of claim 13, further comprising adapting the use of the fluid conduit for the treatment of hydrocephalus.

15. The method of claim 12, wherein the signal comprises an external magnetic signal.

16. The method of claim 12, wherein a device that generates the signal is placed in proximity to the head of a patient.

17. The method of claim 12, further comprising coating the fluid conduit with a composition that inhibits the accumulation of materials at the orifice of the fluid conduit.

18. The method of claim 12, wherein the actuator alternates in a manner comprising a sweeping, vibrating, or rotating motion.

19. A method of manufacturing a fluid conduit having an actuator for use in an implantable medical device comprising:
providing a base substrate of the fluid conduit;
coupling an actuator to the substrate of the fluid conduit,
wherein the actuator is coupled to the fluid conduit so as to allow the actuator to alternate between a first and a second position in response to a signal in a manner that inhibits accumulation of materials or remove materials at an orifice of the fluid conduit when the medical device is implanted in vivo.

20. The method of claim 19, wherein the actuator is manufactured using microelectromechanical systems (MEMS) techniques.
